(19) United States
(12) Patent Application Publication
(10) Pub. No.: US 2025/0382275 A1
(43) Pub. Date: Dec. 18, 2025

(54) METHOD FOR PRODUCING FLUORINE-CONTAINING DIOXOLANE, AND COMPOSITION USEFUL FOR PRODUCING SAME

(71) Applicant: DAIKIN INDUSTRIES, LTD., Osaka (JP)

(72) Inventors: Yuki SUZUKI, Osaka (JP); Yoshichika KUROKI, Osaka (JP); Atsushi SHIRAI, Osaka (JP); Daichi MARI, Osaka (JP); Noriyuki SHINOKI, Osaka (JP); Michiaki OKADA, Osaka (JP); Tadashi INO, Osaka (JP)

(73) Assignee: DAIKIN INDUSTRIES, LTD., Osaka (JP)

(21) Appl. No.: 19/303,742

(22) Filed: Aug. 19, 2025

Related U.S. Application Data
(63) Continuation of application No. PCT/JP2024/005606, filed on Feb. 16, 2024.

(30) Foreign Application Priority Data
Feb. 22, 2023 (JP) 2023-026620

(51) Int. Cl. C07D 317/42; C07D 317/16; C07D 317/34

(52) U.S. Cl. CPC C07D 317/42; C07D 317/16; C07D 317/34

(57) ABSTRACT

An object of the present disclosure is to provide a novel method for producing a 1,3-dioxolane compound having a 2-(difluoromethylene) structure (e.g., 2-(difluoromethylene)-4,4,5-trifluoro-5-(trifluoromethyl)-1,3-dioxolane) from a corresponding carboxylic acid salt, the method suppressing the production of HF adducts (e.g., 2-hydro-perfluoro(2,4-dimethyl-1,3-dioxolane)) as by-products. The present disclosure relates to a method for producing a compound represented by formula (1), the method comprising the following steps A, B, C, and D:
step A of reacting a compound represented by formula (2) with at least one base selected from the group consisting of hydroxides, carbonates, and alkoxides of alkali metals and alkaline earth metals to produce a compound represented by formula (3), thereby obtaining a reaction product having a pH range of more than 11.0;
step B of adding carbon dioxide gas to the reaction product obtained in step A, or a liquid obtained by mixing the reaction product with water, to adjust the pH to 6.0 to 11.0, thereby obtaining a pH-adjusted liquid;
step C of concentrating the pH-adjusted liquid obtained in step B to obtain a concentrate; and
step D of heating the concentrate obtained in step C to thermally decompose the compound represented by formula (3), thereby producing the compound represented by formula (1):

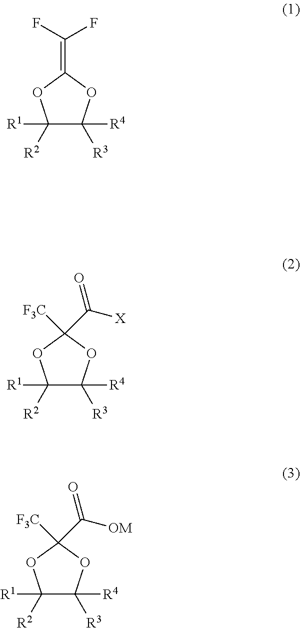

wherein R¹ to R⁴ are each independently a fluorine atom or a C1-C7 fluoroalkyl group optionally containing ethereal oxygen, X is a hydroxy group, a fluorine atom, a chlorine atom, or a C1-C3 alkoxy group in which one or more hydrogen atoms are optionally replaced by fluorine atoms, and M is an alkali metal atom or an alkaline earth metal atom.

### TECHNICAL FIELD

The present disclosure relates to a method for producing fluorine-containing dioxolane and a composition useful for producing the same.

### BACKGROUND ART

1,3-dioxolane compounds having a 2-(difluoromethylene) structure, such as perfluoro(2-methylene-4-methyl-1,3-dioxolane), are used as raw material monomers for fluororesins constituting optical fibers. As a method for producing such perfluorodioxolane, for example, there is a known method in which perfluoro(3,6-bismethyl-1,4-dioxane-2-one) is used as a raw material and converted into a 2-COF compound, a 2-carboxylic acid salt, or a 2-methylene compound, as shown in the following formula (e.g., PTL 1 and PTL 2):

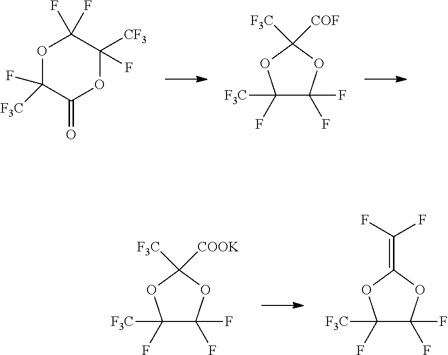

In this method, a carbonyl fluoride compound is generally saponified with a base to convert it into a carboxylic acid salt, which is then thermally decomposed by heating to produce a desired difluoromethylene compound. However, if water, which is a proton source, is present during the thermal decomposition of the carboxylic acid salt obtained from the carbonyl fluoride compound, not only the desired difluoromethylene compound, but also HF adducts in which HF is added to the double bond in the difluoromethylene compound (e.g., 2-hydro-perfluoro(2,4-dimethyl-1,3-dioxolane) shown below), are produced:

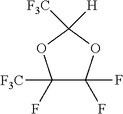 [SMILES: FC(F)(F)C1OC(F)(F)C(F)(C(F)(F)F)O1]

As a result, the yield of the target product decreases.

Furthermore, since the boiling point of the HF adducts is close to that of the target difluoromethylene compound, it is not easy to separate the HF adducts from the target difluoromethylene compound by distillation.

For this reason, attention has been focused on reducing the amount of water that coexists with carboxylic acid salts. For example, PTL 3 states the following:

“The inventors conducted extensive studies regarding a method of producing perfluoro(2-methylene-4-methyl-1,3-dioxolane) and, as a result, newly found that, with at least one of the raw material perfluoro(2,4-dimethyl-2-fluoroformyl-1,3-dioxolane) and a hydrolysis product thereof being reacted with a basic aqueous solution containing one or more cations selected from the group consisting of alkali metal ions and alkaline earth metal ions, a liquid containing the produced perfluoro(2,4-dimethyl-1,3-dioxolane-2-yl)carboxylic acid alkali metal salts or perfluoro(2,4-dimethyl-1,3-dioxolane-2-yl)carboxylic acid alkaline earth metal salts are then separated by a liquid separation operation, one or more water content reduction treatments selected from the group consisting of water evaporation and water adsorption are then performed to be, after that, used in a decarboxylation reaction in a liquid phase system, whereby perfluoro(2-methylene-4-methyl-1,3-dioxolane) can be obtained with a high yield while suppressing production of 2-hydro-perfluoro(2,4-dimethyl-1,3-dioxolane), which is difficult to separate off.”

### CITATION LIST

### Patent Literature

PTL 1: WO2020/166632

PTL 2: WO2020/230822

PTL 3: WO2020/095915

### SUMMARY

The present disclosure typically includes the following embodiments.

A method for producing a compound represented by formula (1):

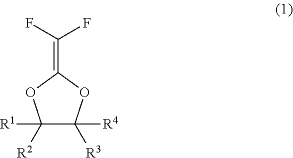

wherein R¹ to R⁴ are each independently a fluorine atom or a C1-C7 fluoroalkyl group optionally containing ethereal oxygen,
the method comprising the following steps A, B, C, and D:
step A of reacting a compound represented by formula (2):

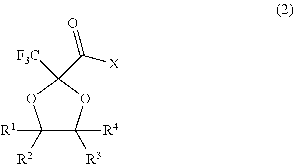

wherein X is a hydroxy group, a fluorine atom, a chlorine atom, or a C1-C3 alkoxy group in which one or more hydrogen atoms are optionally replaced by fluorine atoms, and R¹ to R⁴ are the same as above, with at least one base selected from the group consisting of hydroxides, carbonates, and alkoxides of alkali metals and alkaline earth metals to produce a compound represented by formula (3):

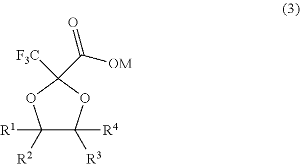

wherein M is an alkali metal atom or an alkaline earth metal atom, and R¹ to R⁴ are the same as above, thereby obtaining a reaction product having a pH range of more than 11.0;
step B of adding carbon dioxide gas to the reaction product obtained in step A, or a liquid obtained by mixing the reaction product with water, to adjust the pH to 6.0 to 11.0, thereby obtaining a pH-adjusted liquid;
step C of concentrating the pH-adjusted liquid obtained in step B to obtain a concentrate; and
step D of heating the concentrate obtained in step C to thermally decompose the compound represented by formula (3), thereby producing the compound represented by formula (1).

### ADVANTAGEOUS EFFECTS

According to the present disclosure, when a corresponding carboxylic acid salt is thermally decomposed to produce a 1,3-dioxolane compound having a 2-(difluoromethylene) structure, the production of the by-product 2-hydro-2-trifluoromethyl compound can be reduced, and a high-purity target product can be produced.

According to the present disclosure, after preparing a carboxylic acid salt, the pH of the reaction liquid is adjusted to 6.0 to 11.0 using carbon dioxide gas, which makes it possible to avoid the use of inorganic acids (sulfuric acid, hydrochloric acid, hydrofluoric acid, etc.) and to prevent corrosion of equipment (e.g., reaction vessels) caused by inorganic acids.

When the composition of the present disclosure is used as a raw material for the production of a 1,3-dioxolane compound having a 2-(difluoromethylene) structure, the production of by-products can be suppressed, and a 1,3-dioxolane compound having a 2-(difluoromethylene) structure can be obtained with high purity.

### DESCRIPTION OF EMBODIMENTS

The above summary of the present disclosure is not intended to describe each disclosed embodiment or every implementation of the present disclosure.

The following description of the present disclosure illustrates embodiments of examples in more detail.

In several parts of the present disclosure, guidance is provided through examples, and these examples can be used in various combinations.

In each case, the group of examples can act as a non-exclusive and representative group.

All publications, patents, and patent applications referred to herein are incorporated herein by reference without modification.

### Terms

Unless otherwise specified, the symbols and abbreviations in the present specification can be understood in the sense commonly used in the technical field to which the present disclosure pertains, according to the context of the present specification.

In the present specification, the terms “contain” and “comprise” are used with the intention to include the terms “consist essentially of” and “consist of.”

Unless otherwise specified, the steps, treatments, or operations described in the present specification can be performed at room temperature.

In the present specification, room temperature can mean a temperature in the range of 10 to 40° C.

In the present specification, the notation “Cn-Cm” (where n and m are numbers) indicates that the number of carbon atoms is n or more and m or less, as is commonly understood by a person skilled in the art.

In the present specification, unless otherwise specified, examples of the “alkyl group” include linear or branched C1-C10 (preferably C1-C7, more preferably C1-C6, even more preferably C1-C4, and particularly preferably C1-C3) alkyl groups, such as methyl, ethyl, propyl (e.g., n-propyl and isopropyl), butyl (e.g., n-butyl, isobutyl, sec-butyl, and tert-butyl), pentyl (e.g., n-pentyl, tert-pentyl, neopentyl, isopentyl, sec-pentyl, and 3-pentyl), hexyl, heptyl, octyl, nonyl, and decyl; and cyclic C3-C10 (e.g., C3-C6, C4-C6, C3-C5, or C5-C6) alkyl groups, such as cyclopropyl, cyclobutyl, cyclopentyl, cyclohexyl, cycloheptyl, cyclooctyl, and adamantyl.

In the present specification, unless otherwise specified, the “alkoxy group” can be a group represented by RO—, wherein R is an alkyl group (e.g., a C1-C10 alkyl group, a C1-C7 alkyl group, a C1-C6 alkyl group, a C1-C4 alkyl group, or a C1-C3 alkyl group).

Examples of the alkoxy group include linear or branched C1-C10 (preferably C1-C7, more preferably C1-C6, even more preferably C1-C4, and particularly preferably C1-C3) alkoxy groups, such as methoxy, ethoxy, propoxy (e.g., n-propoxy and isopropoxy), butoxy (e.g., n-butoxy, isobutoxy, sec-butoxy, and tert-butoxy), pentyloxy (e.g., n-pentyloxy, tert-pentyloxy, neopentyloxy, isopentyloxy, sec-pentyloxy, and 3-pentyloxy), hexyloxy, heptyloxy, octyloxy, nonyloxy, and decyloxy; and cyclic C3-C10 (e.g., C3-C6, C4-C6, C3-C5, or C5-C6) alkoxy groups, such as cyclopropoxy, cyclobutoxy, cyclopentyloxy, cyclohexyloxy, cycloheptyloxy, cyclooctyloxy, and adamantyloxy.

In the present specification, unless otherwise specified, the “fluoroalkyl group” is an alkyl group in which at least one hydrogen atom is replaced by a fluorine atom, and also includes a perfluoroalkyl group in which all of the hydrogen atoms in the alkyl group are replaced by fluorine atoms.

The number of carbon atoms in the fluoroalkyl group can be, for example, 1 to 10, 1 to 7, 1 to 6, 1 to 5, 1 to 4, 1 to 3, 1 or 2, or 1.

Examples of the fluoroalkyl group include linear or branched C1-C10 (preferably C1-C7, more preferably C1-C6, even more preferably C1-C4, and particularly preferably C1-C3) fluoroalkyl groups (preferably perfluoroalkyl groups), such as methyl having 1 to 3 fluorine atoms, ethyl having 1 to 5 fluorine atoms, propyl having 1 to 7 fluorine atoms (e.g., n-propyl and isopropyl), butyl having 1 to 9 fluorine atoms (e.g., n-butyl, isobutyl, sec-butyl, and tert-butyl), pentyl having 1 to 11 fluorine atoms (e.g., n-pentyl, tert-pentyl, neopentyl, isopentyl, sec-pentyl, and 3-pentyl), hexyl having 1 to 13 fluorine atoms, heptyl having 1 to 15 fluorine atoms, octyl having 1 to 17 fluorine atoms, nonyl having 1 to 19 fluorine atoms, and decyl having 1 to 21 fluorine atoms.

The number of fluorine atoms contained in the fluoroalkyl group may be one to the maximum substitutable number, and can be, for example, 1 to 21, 1 to 19, 1 to 17, 1 to 15, 1 to 13, 1 to 11, 1 to 9, 1 to 7, 1 to 5, or 1 to 3.

In the present specification, unless otherwise specified, the “fluoroalkoxy group” is an alkoxy group in which at least one hydrogen atom is replaced by a fluorine atom, and also includes a perfluoroalkoxy group in which all of the hydrogen atoms in the alkoxy group are replaced by fluorine atoms.

The number of carbon atoms in the fluoroalkoxy group can be, for example, 1 to 10, 1 to 7, 1 to 6, 1 to 5, 1 to 4, 1 to 3, 1 or 2, or 1.

Examples of the fluoroalkoxy group include linear or branched C1-C10 (preferably C1-C7, more preferably C1-C6, even more preferably C1-C4, and particularly preferably C1-C3) fluoroalkoxy groups (preferably perfluoroalkoxy groups), such as methoxy having 1 to 3 fluorine atoms, ethoxy having 1 to 5 fluorine atoms, propoxy having 1 to 7 fluorine atoms (e.g., n-propoxy and isopropoxy), butoxy having 1 to 9 fluorine atoms (e.g., n-butoxy, isobutoxy, sec-butoxy, and tert-butoxy), pentyloxy having 1 to 11 fluorine atoms (e.g., n-pentyloxy, tert-pentyloxy, neopentyloxy, isopentyloxy, sec-pentyloxy, and 3-pentyloxy), hexyloxy having 1 to 13 fluorine atoms, heptyloxy having 1 to 15 fluorine atoms, octyloxy having 1 to 17 fluorine atoms, nonyloxy having 1 to 19 fluorine atoms, and decyloxy having 1 to 21 fluorine atoms.

The number of fluorine atoms contained in the fluoroalkoxy group may be one to the maximum substitutable number, and can be, for example, 1 to 21, 1 to 19, 1 to 17, 1 to 15, 1 to 13, 1 to 11, 1 to 9, 1 to 7, 1 to 5, or 1 to 3.

In the present specification, unless otherwise specified, the “C1-C7 fluoroalkyl group optionally containing ethereal oxygen” includes the above fluoroalkyl groups having 1 to 7 carbon atoms, and C1-C7 fluoroalkyl groups containing ethereal oxygen. Further, the “C1-C7 fluoroalkyl group optionally containing ethereal oxygen” includes a perfluoro C1-C7 alkyl group optionally containing ethereal oxygen in which all of the hydrogen atoms in the alkyl group are replaced by fluorine atoms.

The C1-C7 fluoroalkyl group containing ethereal oxygen includes a C1-C7 fluoroalkyl group (preferably a perfluoro C1-C7 alkyl group) having ethereal oxygen “—O—” at its end or inside. Therefore, the C1-C7 fluoroalkyl group containing ethereal oxygen can also be referred to as a group having an ethereal oxygen atom at the end or between the carbon-carbon bonds of the C1-C7 fluoroalkyl group. Trifluoromethoxy (CF₃—O—) is an example of a fluoroalkyl group having “—O—” at the end, and perfluoro(methoxy methyl) (CF₃—O—CF₂—) is an example of a fluoroalkyl group having “—O—” inside the structure.

The number of ethereal oxygen atoms contained in the C1-C7 fluoroalkyl group containing ethereal oxygen can be 1, 2, 3, or the like, but is preferably 1 or 2, and preferably 1.

The number of carbon atoms in the C1-C7 fluoroalkyl group containing ethereal oxygen is 1 to 7, preferably 1 to 6, more preferably 1 to 4, and even more preferably 1 to 3.

The C1-C7 fluoroalkyl group containing ethereal oxygen includes a C1-C7 fluoroalkoxy group, a C1-C6 fluoroalkoxytrifluoromethyl group, a C1-C5 fluoroalkoxypentafluoroethyl group, and the like.

Examples of the C1-C7 fluoroalkoxy group include methoxy having 1 to 3 fluorine atoms, ethoxy having 1 to 5 fluorine atoms, propoxy having 1 to 7 fluorine atoms (e.g., n-propoxy and isopropoxy), butoxy having 1 to 9 fluorine atoms (e.g., n-butoxy, isobutoxy, sec-butoxy, and tert-butoxy), pentyloxy having 1 to 11 fluorine atoms (e.g., n-pentyloxy, tert-pentyloxy, neopentyloxy, isopentyloxy, sec-pentyloxy, and 3-pentyloxy), hexyloxy having 1 to 13 fluorine atoms, heptyloxy having 1 to 15 fluorine atoms, and the like.

The C1-C6 fluoroalkoxytrifluoromethyl group is C1-C6 fluoroalkoxy —CF₂—. Examples of C1-C6 fluoroalkoxy are listed in the examples of “fluoroalkoxy groups” described above.

The C1-C5 fluoroalkoxypentafluoroethyl group is C1-C5 fluoroalkoxy —C₂F₄—. Examples of C1-C5 fluoroalkoxy are listed in the examples of “fluoroalkoxy groups” described above.

In the present specification, unless otherwise specified, examples of the “C1-C3 alkyl group in which one or more hydrogen atoms are optionally replaced by fluorine atoms” include a C1-C3 alkyl group and a C1-C3 alkyl group in which one or more hydrogen atoms are replaced by fluorine atoms. In other words, the “C1-C3 alkyl group in which one or more hydrogen atoms are optionally replaced by fluorine atoms” includes a C1-C3 alkyl group and a C1-C3 fluoroalkyl group. Further, the C1-C3 alkyl group in which one or more hydrogen atoms are replaced by fluorine atoms includes a perfluoro C1-C3 alkyl group in which all of the hydrogen atoms in the alkyl group are replaced by fluorine atoms.

Examples of the C1-C3 alkyl group and C1-C3 fluoroalkyl group are listed in the examples of alkyl groups and the examples of fluoroalkyl groups described above.

In the present specification, unless otherwise specified, examples of the “C1-C3 alkoxy group in which one or more hydrogen atoms are optionally replaced by fluorine atoms” include a C1-C3 alkoxy group and a C1-C3 alkoxy group in which one or more hydrogen atoms are replaced by fluorine atoms. In other words, the “C1-C3 alkoxy group in which one or more hydrogen atoms are optionally replaced by fluorine atoms” include a C1-C3 alkoxy group and a C1-C3 fluoroalkoxy group. Further, the C1-C3 alkoxy group in which one or more hydrogen atoms are replaced by fluorine atoms includes a perfluoro C1-C3 alkoxy group in which all of the hydrogen atoms in the alkoxy group are replaced by fluorine atoms.

Examples of the C1-C3 alkoxy group and C1-C3 fluoroalkoxy group are listed in the examples of alkoxy groups and the examples of fluoroalkoxy groups described above.

In the present specification, unless otherwise specified, examples of the “alkali metal” include lithium, sodium, potassium, rubidium, cesium, and francium, with preferred examples including lithium, sodium, and potassium, and more preferred examples including sodium and potassium.

In the present specification, unless otherwise specified, examples of the “alkaline earth metal” include beryllium, magnesium, calcium, strontium, barium, and radium, with preferred examples including magnesium and calcium.

### Method for Producing Compound Represented by Formula (1)

An embodiment of the present disclosure is a method for producing a compound represented by formula (1):

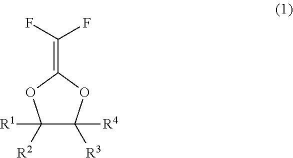

wherein R¹ to R⁴ are each independently a fluorine atom or a C1-C7 fluoroalkyl group optionally containing ethereal oxygen (in the present specification, also referred to as “compound (1)”).

The production method includes steps A, B, C, and D.

Steps A, C, and D, excluding step B, may be performed by a known method, for example, according to the method disclosed in PTL 1, PTL 2, JP2005-002014A, U.S. Pat. No. 3,308,107B, or U.S. Pat. No. 6,664,431B. These publications are incorporated herein by reference.

R¹ to R⁴ can be each independently a fluorine atom, a linear or branched C1-C7 fluoroalkyl group, or a linear or branched C1-C7 fluoroalkyl group containing ethereal oxygen.

R¹ to R⁴ can be each independently a fluorine atom, a linear or branched C1-C6 fluoroalkyl group, or a linear or branched C1-C6 fluoroalkyl group containing ethereal oxygen.

R¹ to R⁴ can be each independently a fluorine atom, a linear or branched C1-C5 fluoroalkyl group, or a linear or branched C1-C5 fluoroalkyl group containing ethereal oxygen.

R¹ to R⁴ can be each independently a fluorine atom, a linear or branched C1-C4 fluoroalkyl group, or a linear or branched C1-C4 fluoroalkyl group containing ethereal oxygen.

R¹ to R⁴ can be each independently a fluorine atom, a linear or branched C1-C3 fluoroalkyl group, or a linear or branched C1-C3 fluoroalkyl group containing ethereal oxygen.

R¹ to R⁴ can be each independently a fluorine atom, a linear or branched perfluoro C1-C7 alkyl group, or a linear or branched perfluoro C1-C7 alkyl group containing ethereal oxygen.

R¹ to R⁴ can be each independently a fluorine atom, a linear or branched perfluoro C1-C6 alkyl group, or a linear or branched perfluoro C1-C6 alkyl group containing ethereal oxygen.

R¹ to R⁴ can be each independently a fluorine atom, a linear or branched perfluoro C1-C5 alkyl group, or a linear or branched perfluoro C1-C5 alkyl group containing ethereal oxygen.

R¹ to R⁴ can be each independently a fluorine atom, a linear or branched perfluoro C1-C4 alkyl group, or a linear or branched perfluoro C1-C4 alkyl group containing ethereal oxygen.

R¹ to R⁴ can be each independently a fluorine atom, a linear or branched perfluoro C1-C3 alkyl group, or a linear or branched perfluoro C1-C3 alkyl group containing ethereal oxygen.

R¹ to R⁴ can be each independently a fluorine atom, a perfluoro C1-C7 alkyl group, or a perfluoro C1-C7 alkoxy group.

R¹ to R⁴ can be each independently a fluorine atom, a perfluoro C1-C6 alkyl group, or a perfluoro C1-C6 alkoxy group.

R¹ to R⁴ can be each independently a fluorine atom, a perfluoro C1-C5 alkyl group, or a perfluoro C1-C5 alkoxy group.

R¹ to R⁴ can be each independently a fluorine atom, a perfluoro C1-C4 alkyl group, or a perfluoro C1-C4 alkoxy group.

R¹ to R⁴ can be each independently a fluorine atom, a perfluoro C1-C3 alkyl group, or a perfluoro C1-C3 alkoxy group.

At least one group of R¹ to R⁴ may be a fluorine atom, and the other groups, when there are a plurality of other groups, may be independently a perfluoro C1-C2 alkyl group or a perfluoro C1-C3 alkoxy group.

At least two groups of R¹ to R⁴ may be fluorine atoms, and the other groups, when there are a plurality of other groups, may be independently a perfluoro C1-C2 alkyl group or a perfluoro C1-C3 alkoxy group.

At least three groups of R¹ to R⁴ may be fluorine atoms, and the other group may be a perfluoro C1-C3 alkyl group or a perfluoro C1-C2 alkoxy group.

At least three groups of R¹ to R⁴ may be fluorine atoms, and the other group may be a perfluoro C1-C3 alkyl group.

All of R¹ to R⁴ may be fluorine atoms.

R¹ may be a trifluoromethyl group or a fluorine atom, and all of R² to R⁴ may be fluorine atoms.

Specific examples of compound (1) include perfluoro(2-methylene-1,3-dioxolane), perfluoro(2-methylene-4-methyl-1,3-dioxolane), perfluoro(2-methylene-4-ethyl-1,3-dioxolane), perfluoro(2-methylene-4,5-dimethyl-1,3-dioxolane), perfluoro(2-methylene-4,5-diethyl-1,3-dioxolane), perfluoro(2-methylene-4-methoxymethyl-1,3-dioxolane), perfluoro(2-methylene-4-ethoxymethyl-1,3-dioxolane), 2-(difluoromethylene)-3a, 4,4,6,6,6a-hexafluorotetrahydrofuro[3,4-d] [1,3]dioxol, and 2-(difluoromethylene)-3a,4,4,5,5,6,6,7,7,7a-decafluorohexahydrobenzo[d] [1,3]dioxol; and preferably perfluoro(2-methylene-1,3-dioxolane) and perfluoro(2-methylene-4-methyl-1,3-dioxolane).

### Step A

In step A, a compound represented by formula (2):

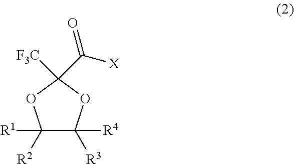

wherein X is a hydroxy group, a fluorine atom, a chlorine atom, or a C1-C3 alkoxy group in which one or more hydrogen atoms are optionally replaced by fluorine atoms, and R¹ to R⁴ are the same as above (in the present specification, also referred to as “compound (2)”), is reacted with at least one base selected from the group consisting of hydroxides, carbonates, and alkoxides of alkali metals and alkaline earth metals to be converted into a compound represented by formula (3):

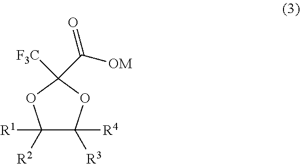

wherein M is an alkali metal atom or an alkaline earth metal atom, and R¹ to R⁴ are the same as above (in the present specification, also referred to as “compound (3)”), thereby obtaining a reaction product having a pH range of more than 11.0.

Compound (2) is a known compound and can be produced, for example, by the method disclosed in PTL 1 or PTL 2, or by appropriately modifying the method.

X can be a hydroxy group, a fluorine atom, a chlorine atom, or a C1-C3 alkoxy group in which one or more hydrogen atoms are optionally replaced by fluorine atoms. X can be a hydroxy group, a fluorine atom, a chlorine atom, methoxy, ethoxy, n-propoxy, i-propoxy, trifluoromethoxy, or 2,2,2-trifluoroethoxy. X can be a hydroxy group, a fluorine atom, methoxy, ethoxy, trifluoromethoxy, or 2,2,2-trifluoroethoxy.

X can be a fluorine atom, a chlorine atom, or a C1-C3 alkoxy group in which one or more hydrogen atoms are optionally replaced by fluorine atoms. X can be a fluorine atom, a chlorine atom, methoxy, ethoxy, n-propoxy, i-propoxy, trifluoromethoxy, or 2,2,2-trifluoroethoxy. X can be a fluorine atom, methoxy, ethoxy, trifluoromethoxy, or 2,2,2-trifluoroethoxy. X can be a fluorine atom, methoxy, ethoxy, n-propoxy, or i-propoxy. X can be a fluorine atom, methoxy, or trifluoromethoxy. X can be a fluorine atom or methoxy.

Specific examples of compound (2) include perfluoro(2-formyl-2,4-dimethyl-1,3-dioxolane), 4,4,5-trifluoro-2,5-bis(trifluoromethyl)-1,3-dioxolane-2-carbonyl chloride, 4,4,5-trifluoro-2,5-bis(trifluoromethyl)-1,3-dioxolane-2-carboxylic acid, 4,4,5-trifluoro-2,5-bis(trifluoromethyl)-1,3-dioxolane-2-carboxylic acid methyl ester, 4,4,5-trifluoro-2,5-bis(trifluoromethyl)-1,3-dioxolane-2-carboxylic acid ethyl ester, 4,4,5-trifluoro-2,5-bis(trifluoromethyl)-1,3-dioxolane-2-carboxylic acid n-propyl ester, 4,4,5-trifluoro-2,5-bis(trifluoromethyl)-1,3-dioxolane-2-carboxylic acid i-propyl ester, 4,4,5-trifluoro-2,5-bis(trifluoromethyl)-1,3-dioxolane-2-carboxylic acid trifluoromethyl ester, and 4,4,5-trifluoro-2,5-bis(trifluoromethyl)-1,3-dioxolane-2-carboxylic acid 2,2,2-trifluoroethyl ester.

Preferred specific examples of compound (2) include perfluoro(2-formyl-2,4-dimethyl-1,3-dioxolane), 4,4,5-trifluoro-2,5-bis(trifluoromethyl)-1,3-dioxolane-2-carboxylic acid, 4,4,5-trifluoro-2,5-bis(trifluoromethyl)-1,3-dioxolane-2-carboxylic acid methyl ester, and 4,4,5-trifluoro-2,5-bis(trifluoromethyl)-1,3-dioxolane-2-carboxylic acid trifluoromethyl ester.

The base includes hydroxides, carbonates, and alkoxides of alkali metals, as well as hydroxides, carbonates, and alkoxides of alkaline earth metals, and may be used singly or in combination of two or more.

Examples of hydroxides include sodium hydroxide, potassium hydroxide, lithium hydroxide, magnesium hydroxide, calcium hydroxide, and barium hydroxide.

Examples of carbonates include sodium carbonate, potassium carbonate, lithium carbonate, magnesium carbonate, calcium carbonate, barium carbonate, sodium hydrogen carbonate, potassium hydrogen carbonate, and lithium hydrogen carbonate.

Examples of alkoxides include sodium methoxide, sodium ethoxide, sodium butoxide, potassium methoxide, potassium ethoxide, potassium butoxide, lithium methoxide, lithium ethoxide, magnesium methoxide, magnesium ethoxide, calcium methoxide, and calcium ethoxide.

The base is preferably at least one member selected from the group consisting of potassium carbonate, sodium carbonate, magnesium carbonate, sodium hydrogen carbonate, potassium hydrogen carbonate, potassium hydroxide, sodium hydroxide, magnesium hydroxide, potassium methoxide, sodium methoxide, magnesium methoxide, potassium ethoxide, sodium ethoxide, and magnesium ethoxide.

The base is more preferably at least one member selected from the group consisting of potassium carbonate, sodium carbonate, magnesium carbonate, potassium hydroxide, and sodium hydroxide.

The base is even more preferably at least one member selected from the group consisting of potassium carbonate, sodium carbonate, potassium hydroxide, and sodium hydroxide.

The base is particularly preferably at least one member selected from the group consisting of potassium carbonate and sodium hydroxide.

Compound (3) is a carboxylic acid salt of compound (2) corresponding to the base used in step A. Specific examples and preferred examples of compound (3) are carboxylic acid salts corresponding to the specific examples and preferred examples of compound (2), respectively.

M can be an alkali metal atom or an alkaline earth metal atom.

M is preferably an alkali metal atom, more preferably a potassium atom or a sodium atom, and even more preferably a potassium atom.

M can be a group corresponding to the base used in step A. Therefore, when the base is a potassium salt, M can be a potassium atom.

Step A may be performed in the presence or absence of a solvent.

As the solvent, water or an organic solvent may be used. Solvents can be used singly or in combination of two or more.

Examples of organic solvents include alkyl alcohol solvents, ether solvents, aromatic solvents, saturated hydrocarbon solvents, nitrile solvents, sulfoxide solvents, and halogenated hydrocarbon solvents.

Preferred examples of organic solvents include alkyl alcohol solvents, ether solvents, halogenated hydrocarbon solvents, and nitrile solvents.

Examples of alkyl alcohol solvents include linear or branched C1-C10 alkyl alcohols, preferably linear or branched C1-5 alkyl alcohols, more preferably linear or branched C1-4 alkyl alcohols, even more preferably methanol, ethanol, n-propanol, isopropanol, n-butyl alcohol, sec-butyl alcohol, or tert-butyl alcohol, and particularly preferably methanol or ethanol.

Preferred specific examples of ether solvents include dimethyl ether, diethylene glycol dimethyl ether, triethylene glycol dimethyl ether, tetraethylene glycol dimethyl ether, diethyl ether, diisopropyl ether, dioxane, tetrahydrofuran, 1,2-dimethoxy ethane, and crown ether.

Preferred specific examples of aromatic solvents include benzene, toluene, and xylene.

Preferred specific examples of saturated hydrocarbon solvents include n-pentane, n-hexane, cyclohexane, and n-heptane.

Preferred specific examples of nitrile solvents include 1,4-dicyanobutane, acetonitrile, and benzonitrile.

Preferred specific examples of sulfoxide solvents include dimethyl sulfoxide and sulfolane.

Preferred specific examples of halogenated hydrocarbon solvents include methylene chloride, chloroform, carbon tetrachloride, 1,2-dichloroethane, 1,2-dichlorobenzene, chlorobenzene, and perfluorohexane.

Preferred specific examples of organic solvents include methanol, ethanol, dimethyl ether (DME), diethylene glycol dimethyl ether, triethylene glycol dimethyl ether, tetraethylene glycol dimethyl ether, 1,4-dicyanobutane, methylene chloride, chloroform, tetrahydrofuran, perfluorohexane, and acetonitrile.

The amount of the base used in step A can be within the range of 0.05 to 10 mol, 0.1 to 10 mol, 0.1 to 5 mol, or the like, preferably 0.5 to 10 mol, and more preferably 0.5 to 5 mol, per mol of compound (2).

The amount of the base is preferably an amount in which the pH of the reaction product obtained in step A or a liquid obtained by mixing the reaction product with water exceeds 11.0.

When a solvent is used in step A, the amount thereof may be an amount that can function as a solvent, based on common technical knowledge etc.

The reaction temperature in step A may be preferably within the range of −50 to 120° C., more preferably −20 to 100° C., and even more preferably −10 to 70° C.

The reaction time in step A is not limited as long as the target substance is produced, but may be preferably within the range of 0.1 hours to 48 hours, more preferably 0.1 hours to 24 hours, and even more preferably 0.1 hours to 12 hours.

In step A, compound (2) is converted into compound (3) to obtain a reaction product containing compound (3).

The reaction product may be an aqueous solution or a non-aqueous solution. The aqueous solution includes an aqueous solution of compound (3). The non-aqueous solution includes an organic solvent containing compound (3) and a solid containing compound (3).

In step A, when water or a mixed solvent of water and other solvents is used as the solvent, the reaction product can be obtained as an aqueous solution of compound (3).

In step A, when an organic solvent is used as the solvent, the reaction product can be obtained as an organic solvent (including slurry) containing compound (3).

In step A, when no solvent is used, the reaction product can be obtained as a solid (including slurry) containing compound (3).

The pH of the reaction product can be in the range of more than 11.0, and can be in the range of 12.0 or more. The reaction product is subjected to subsequent step B.

### Step B

Step B is a step of adjusting the pH of the reaction product having a pH range of more than 11.0 (which can be a liquid) obtained in step A to a range of 6.0 to 11.0 using carbon dioxide gas before subjecting it to the subsequent concentration step, step C. The pH may be 6.0 to 10.0 or 6.5 to 10.0, and is preferably 6.5 to 9.0, more preferably 6.5 to 8.5, and particularly preferably 6.9 to 7.9. Before pH adjustment, the pH of the reaction product obtained in step A may be measured in order to confirm that the pH of the reaction product exceeds 11.0. The reaction product whose pH has been adjusted (also referred to as “the pH-adjusted liquid”) is subjected to step C.

pH measurement is explained.

The pH may be a value determined using a pH test paper or a pH meter (e.g., Benchtop pH/Water Quality Analyzer F-74, produced by Horiba, Ltd.), or by titration using a pH indicator, and is preferably a value determined using a pH meter.

The reaction product obtained in step A may be a liquid or a solid. In pH measurement to confirm the pH of the reaction product obtained in step A, it is not necessary to measure the pH of the entire reaction product obtained in step A, and it is acceptable to measure the pH of a portion of the reaction product.

When the reaction product obtained in step A is an aqueous solution of compound (3), a portion or all of this aqueous solution can be subjected to pH measurement.

When the reaction product obtained in step A is an organic solvent containing compound (3) (including an organic solvent solution in which compound (3) is dissolved) (e.g., an alcohol solution in which compound (3) is dissolved; the same applies below), a mixed liquid obtained by mixing a portion or all of this organic solvent with water can be subjected to pH measurement. When the mixed liquid separates into an aqueous phase and an organic phase after being allowed to stand, the aqueous phase can be subjected to pH measurement.

When the reaction product obtained in step A is a solid containing compound (3), a mixed liquid in which compound (3) is dissolved by mixing a portion or all of this solid with water can be subjected to pH measurement.

Carbon dioxide gas is used for pH measurement. The use of carbon dioxide gas can suppress the production of HF adducts in which HF is added to the double bond in a difluoromethylene compound (e.g., 2-hydro-perfluoro(2,4-dimethyl-1,3-dioxolane)) as by-products, compared to other pH adjustment treatments (e.g., acidic solution treatment).

The target of carbon dioxide gas treatment is not limited as long as its pH can be adjusted by carbon dioxide gas treatment. The reaction product may be subjected to pH adjustment treatment as it is, a filtrate obtained by filtering the reaction product may be subjected to pH adjustment treatment, or a concentrate obtained by concentrating the reaction product may be subjected to pH adjustment treatment.

When the reaction product obtained in step A is an aqueous solution of compound (3), this aqueous solution can be subjected to carbon dioxide gas treatment.

When the reaction product obtained in step A is an organic solvent solution in which compound (3) is dissolved, this organic solvent solution can be subjected to carbon dioxide gas treatment.

When the reaction product obtained in step A is an organic solvent containing compound (3), a mixed liquid obtained by mixing this organic solvent with water, or an aqueous phase obtained by separating the mixed liquid, can be subjected to carbon dioxide gas treatment.

When the reaction product obtained in step A is a solid containing compound (3), a liquid in which compound (3) is dissolved by mixing this solid with water or an organic solvent can be subjected to carbon dioxide gas treatment.

Examples of the method of carbon dioxide gas treatment include bubbling that supplies carbon dioxide gas into the target liquid, a method of injecting carbon dioxide gas into the target liquid in a sealed container, a method of adding dry ice (which may be in powder or solid form) during the reaction and utilizing the evaporated carbon dioxide gas, and other methods, but are not limited thereto as long as the pH of the target object can be adjusted to the desired range using carbon dioxide gas.

The conditions of the carbon dioxide gas treatment are not limited as long as the pH of the target object can be adjusted to the desired range.

The amount of carbon dioxide gas added can be, for example, 100 to 10000 mol % or 300 to 10000 mol %, preferably 300 to 6000 mol %, even more preferably 500 to 6000 mol %, and particularly preferably 600 to 5000 mol %, based on the content of the compound represented by formula (3) in the reaction product obtained in step A.

The temperature of the carbon dioxide gas treatment can be, for example, −100 to 100° C., and preferably −10 to 40° C.

The time of the carbon dioxide gas treatment can be, for example, 0.1 to 72 hours, and preferably 0.5 to 24 hours.

The carbon dioxide gas treatment can be performed under increased pressure, reduced pressure, or atmospheric pressure.

The pH-adjusted liquid obtained by the carbon dioxide gas treatment is subjected to subsequent step C.

### Step C

In step C, the pH-adjusted liquid obtained in step B is concentrated.

The concentration step can be performed according to a known concentration method. Examples of concentration methods include vacuum distillation, drying, extraction, precipitation, distillation, chromatography, and the like, which can be used singly or in combination of two or more. Preferred concentration methods are vacuum distillation and drying.

Examples of concentration conditions include vacuum distillation at 0 to 150° C. and drying at 0 to 150° C.

The concentrate obtained by concentration can be in the form of a liquid, gel, or solid.

In the concentration step, the pH-adjusted liquid is concentrated until the concentration of compound (3) reaches, for example, 90 mass % or more, 92 mass % or more, or 94 mass % or more.

### Step D

In step D, the concentrate obtained in step C is heated. By heating the concentrate, compound (3) contained in the concentrate is decarboxylated to produce compound (1) in high purity. Step D can be performed in the reactor in which the concentration treatment of step C has been performed.

Step D may be performed in an organic solvent or without a solvent. The organic solvent is not limited as long as it can decarboxylate compound (3). The organic solvent may be any solvent that is known to be usable for the decarboxylation of compound (3). Examples and preferred examples of the organic solvent are the same as those of the organic solvent in step A. Preferred specific examples of the organic solvent include 1,2-dimethoxyethane, diethylene glycol dimethyl ether, triethylene glycol dimethyl ether, tetraethylene glycol dimethyl ether, 1,4-dicyanobutane, and acetonitrile.

The amount of the organic solvent used in step D may be an amount that can function as a solvent, based on common technical knowledge etc.

When the object to be heated contains a solvent, the reaction temperature in step D may be preferably within the range of 50 to 400° C., more preferably 70 to 300° C., and even more preferably 100 to 200° C. When the object to be heated does not contain a solvent, the reaction temperature is preferably within the range of 100 to 400° C., more preferably 150 to 400° C., and even more preferably 150 to 350° C.

The reaction time in step D is not limited as long as compound (1) is produced, but may be preferably within the range of 0.1 hours to 48 hours, more preferably 0.1 hours to 24 hours, and even more preferably 0.1 hours to 12 hours.

The reaction in step D may be performed in the presence or absence of an inert gas (e.g., nitrogen gas), and preferably in the absence of an inert gas.

Step D can be performed under reduced pressure, atmospheric pressure, or increased pressure.

Compound (1) produced in step D can be isolated or purified, if desired, by a conventional method, such as extraction, dissolution, concentration, precipitation, dehydration, adsorption, distillation, rectification, or chromatography, or a combination of these methods.

### Composition (1)

An embodiment of the present disclosure is a composition comprising:
at least one compound selected from the group consisting of a compound represented by formula (3):

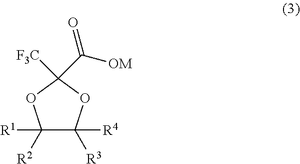

wherein R¹ to R⁴ are each independently a fluorine atom or a C1-C7 fluoroalkyl group optionally containing ethereal oxygen, and M is an alkali metal atom or an alkaline earth metal atom; and a compound represented by formula (4):

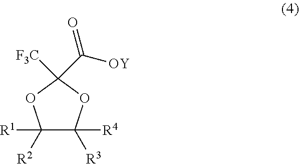

wherein Y is a hydrogen atom or a C1-C3 alkyl group in which one or more hydrogen atoms are optionally replaced by fluorine atoms, and R¹ to R⁴ are the same as above (in the present specification, also referred to as “compound (4)”); and
carbon dioxide gas,
   wherein when the composition is an aqueous solution of at least one compound selected from the group consisting of compounds (3) and (4), the pH of the composition (aqueous solution) is within the range of 6.0 to 11.0, and when the composition is a non-aqueous solution, the pH of an aqueous solution obtained by adding water to the composition (non-aqueous solution) is within the range of 6.0 to 11.0 (in the present specification, also referred to as “composition (1)”).

The composition may be an aqueous solution or a non-aqueous solution. The aqueous solution includes an aqueous solution of at least one compound selected from the group consisting of compounds (3) and (4). The non-aqueous solution includes an organic solvent containing at least one compound selected from the group consisting of compounds (3) and (4) (including an organic solvent solution in which at least one compound selected from the group consisting of compounds (3) and (4) is dissolved (e.g., an alcohol solution in which at least one compound selected from the group consisting of compounds (3) and (4) is dissolved); the same applies below).

When the composition is an aqueous solution, its pH is within the range of 6.0 to 11.0, and when the composition is a non-aqueous solution, the pH of an aqueous solution prepared by adding water to the composition can be within the range of 6.0 to 11.0. These pHs may both be 6.0 to 10.0 or 6.5 to 10.0, and are preferably 6.5 to 9.0, even more preferably 6.5 to 8.5, and particularly preferably 6.9 to 7.9.

When the non-aqueous solution is an organic solvent solution in which at least one compound selected from the group consisting of compounds (3) and (4) is dissolved, the pH of the solution can be within the range of 6.0 to 11.0. The pH may be 6.0 to 10.0 or 6.5 to 10.0, and is preferably 6.5 to 9.0, even more preferably 6.5 to 8.5, and particularly preferably 6.9 to 7.9.

When the non-aqueous solution is an organic solvent containing at least one compound selected from the group consisting of compounds (3) and (4), the pH of a mixed liquid obtained by mixing this organic solvent with water, or an aqueous phase obtained by separating the mixed liquid, can be within the range of 6.0 to 11.0. The pH may be 6.0 to 10.0 or 6.5 to 10.0, and is preferably 6.5 to 9.0, even more preferably 6.5 to 8.5, and particularly preferably 6.9 to 7.9.

Y can be a hydrogen atom or a C1-C3 alkyl group in which one or more hydrogen atoms are optionally replaced by fluorine atoms.

Y can be a hydrogen atom, a C1-C3 alkyl group, or a perfluoro C1-C3 alkyl group.

Y can be a hydrogen atom or a C1-C3 alkyl group in which one or more hydrogen atoms are replaced by fluorine atoms.

Y can be a hydrogen atom or a C1-C3 alkyl group.

Y can be a hydrogen atom or a perfluoro C1-C3 alkyl group.

Examples of Y include a hydrogen atom, methyl, ethyl, n-propyl, i-propyl, trifluoromethyl, 2,2,2-trifluoroethyl, pentafluoroethyl, perfluoro-n-propyl, and perfluoro-i-propyl.

Y may be a hydrogen atom, methyl, ethyl, n-propyl, i-propyl, trifluoromethyl, or 2,2,2-trifluoroethyl.

Y may be a hydrogen atom, methyl, ethyl, or trifluoromethyl.

Y may be methyl, ethyl, n-propyl, i-propyl, trifluoromethyl, or 2,2,2-trifluoroethyl.

Y can be methyl, ethyl, n-propyl, or i-propyl.

Y can be methyl.

For the details of compounds (3) and (4), R¹ to R⁴, and M, the above explanations for these are applied.

Specific examples of compound (4) include 4,4,5-trifluoro-2,5-bis(trifluoromethyl)-1,3-dioxolane-2-carboxylic acid, 4,4,5-trifluoro-2,5-bis(trifluoromethyl)-1,3-dioxolane-2-carboxylic acid methyl ester, 4,4,5-trifluoro-2,5-bis(trifluoromethyl)-1,3-dioxolane-2-carboxylic acid ethyl ester, 4,4,5-trifluoro-2,5-bis(trifluoromethyl)-1,3-dioxolane-2-carboxylic acid n-propyl ester, 4,4,5-trifluoro-2,5-bis(trifluoromethyl)-1,3-dioxolane-2-carboxylic acid i-propyl ester, 4,4,5-trifluoro-2,5-bis(trifluoromethyl)-1,3-dioxolane-2-carboxylic acid trifluoromethyl ester, and 4,4,5-trifluoro-2,5-bis(trifluoromethyl)-1,3-dioxolane-2-carboxylic acid 2,2,2-trifluoroethyl ester. Preferred specific examples of compound (4) include 4,4,5-trifluoro-2,5-bis(trifluoromethyl)-1,3-dioxolane-2-carboxylic acid, 4,4,5-trifluoro-2,5-bis(trifluoromethyl)-1,3-dioxolane-2-carboxylic acid methyl ester, and 4,4,5-trifluoro-2,5-bis(trifluoromethyl)-1,3-dioxolane-2-carboxylic acid trifluoromethyl ester.

Since compound (3) is produced by step A described above, composition (1) can be the pH-adjusted liquid after the carbon dioxide gas treatment described above. Therefore, this composition can be produced, for example, by step B described above. Alternatively, this composition can also be produced by mixing at least one compound selected from the group consisting of compounds (3) and (4), and carbon dioxide gas.

Composition (1) may contain at least one compound selected from the group consisting of compounds (3) and (4) in an amount of, for example, 0.1 to 99.9 mass %, preferably 1 to 90 mass, and more preferably 5 to 80 mass %, based on the mass of composition (1).

When composition (1) contains compounds (3) and (4), the content ratio of compound (3) can be, for example, 0.1 to 99.9 mass %, preferably 50 to 99.9 mass %, and more preferably 75 to 99.9 mass, based on the total of compounds (3) and (4).

Composition (1) may contain carbon dioxide gas in an amount of, for example, 0.05 to 1000 mass % or 0.1 to 500 mass %, preferably 0.1 to 100 mass %, more preferably 0.2 to 50 mass %, even more preferably 0.2 to 30 mass %, and particularly preferably 0.3 to 17 mass %, based on the mass of composition (1).

Composition (1) may contain water and/or an organic solvent. For the details of the organic solvent, the description of the organic solvent explained in the production method of the present disclosure can be applied.

Composition (1) may contain water in an amount of, for example, 5 to 90 mass %, preferably 10 to 80 mass %, and more preferably 10 to 70 mass %, based on the mass of composition (1).

Composition (1) may contain an organic solvent in an amount of, for example, 5 to 90 mass %, preferably 10 to 80 mass %, and more preferably 10 to 70 mass %, based on the mass of composition (1).

Composition (1) may contain other components in addition to at least one compound selected from the group consisting of compounds (3) and (4), and carbon dioxide gas. Examples of other components include bases, alkali metal or alkaline earth metal salts of hydrogen halides, alkali metal or alkaline earth metal salts of inorganic acids, and the like. The description of the base in the production method of the present disclosure can be applied to the base. Composition (1) may contain other components in an amount of, for example, 0.1 to 30 mass % or 0.1 to 20 mass %, based on the mass of composition (1).

Composition (1) is concentrated in step C, and the resulting concentrate is heated in step D to decarbonate compound (3), producing high-purity compound (1). In this case, the production of HF adducts, which are by-products and are difficult to separate from compound (1), is reduced. This is presumably because composition (1) contains carbon dioxide gas, and the pH of composition (1) is within the range of 6.0 to 11.0, 6.0 to 10.0, 6.5 to 10.0, 6.5 to 9.0, 6.5 to 8.5, or 6.9 to 7.9. Composition (1) is useful as a supply source of compound (1).

### Composition (2)

An embodiment of the present disclosure is a composition comprising:
at least one compound selected from the group consisting of a compound represented by formula (3):

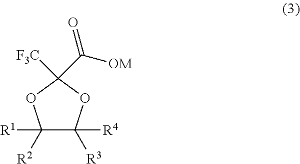

wherein R¹ to R⁴ are each independently a fluorine atom or a C1-C7 fluoroalkyl group optionally containing ethereal oxygen, and M is an alkali metal atom or an alkaline earth metal atom; and a compound represented by formula (4):

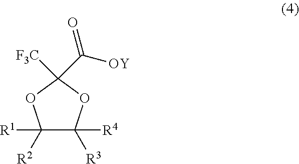

wherein Y is a hydrogen atom or a C1-C3 alkyl group in which one or more hydrogen atoms are optionally replaced by fluorine atoms, and R¹ to R⁴ are the same as above; and
carbon dioxide gas,
   wherein the content of carbon dioxide gas is 0.05 to 1000 mass % based on the mass of the composition (in the present specification, also referred to as “composition (2)”).

For the details of compounds (3) and (4), R¹ to R⁴, M, and Y, the above explanations for these are applied.

Since compound (3) is produced by step A described above, composition (1) can be the pH-adjusted liquid after the carbon dioxide gas treatment or the concentrate after the concentration treatment described above. Therefore, this composition can be produced, for example, by step B or C described above. Alternatively, this composition can also be produced by mixing at least one compound selected from the group consisting of compounds (3) and (4), and carbon dioxide gas.

Composition (2) may contain at least one compound selected from the group consisting of compounds (3) and (4) in an amount of, for example, 1 to 99.9 mass %, preferably 10 to 99.9 mass %, and more preferably 50 to 99.9 mass %, based on the content of composition (2).

When composition (2) contains compounds (3) and (4), the content ratio of compound (3) can be, for example, 0.1 to 99.9 mass %, preferably 50 to 99.9 mass %, and more preferably 75 to 99.9 mass %, based on the total of compounds (3) and (4).

Composition (2) may contain carbon dioxide gas in an amount of, for example, 0.1 to 10000 mol %, preferably 0.1 to 7000 mol %, more preferably 0.1 to 5000 mol %, and even more preferably 1 to 5000 mol %, based on the content of compound (3).

Composition (2) may contain carbon dioxide gas in an amount of, for example, 0.05 to 1000 mass % or 0.1 to 500 mass %, preferably 0.1 to 100 mass %, more preferably 0.2 to 50 mass %, even more preferably 0.2 to 30 mass %, and particularly preferably 0.3 to 17 mass %, based on the content of composition (2).

Composition (2) may contain water and/or an organic solvent. For the details of the organic solvent, the description of the organic solvent explained in the production method of the present disclosure can be applied.

Composition (2) may contain water in an amount of, for example, 0.001 to 90 mass %, preferably 0.001 to 80 mass %, and more preferably 0.001 to 70 mass %, based on the mass of composition (2).

Composition (2) may contain an organic solvent in an amount of, for example, 0.001 to 90 mass %, preferably 0.001 to 80 mass %, and more preferably 0.001 to 70 mass %, based on the mass of composition (2).

Composition (2) may contain other components in addition to at least one compound selected from the group consisting of compounds (3) and (4), and carbon dioxide gas. Examples of other components include bases, alkali metal or alkaline earth metal salts of hydrogen halides, alkali metal or alkaline earth metal salts of inorganic acids, and the like. The description of the base in the production method of the present disclosure can be applied to the base. Composition (2) may contain other components in an amount of, for example, 0.1 to 30 mass % or 0.1 to 20 mass %, based on the mass of composition (2).

Composition (2) is concentrated in step C, and the resulting concentrate is heated in step D, or when composition (2) is sufficiently concentrated, composition (2) is heated in step D, to decarbonate compound (3), producing high-purity compound (2). In this case, the production of HF adducts, which are by-products and are difficult to separate from compound (1), is reduced. This is presumably because composition (2) contains carbon dioxide gas and the content of carbon dioxide gas is 0.05 to 1000 mass based on the mass of composition (2). Composition (2) is useful as a supply source of compound (1).

In the present disclosure, the amount of carbon dioxide gas can be determined by the Orsat method, a gas absorption method, an electrical conductivity method, a thermal conductivity method, an infrared absorption method, a gas chromatography method, a mass spectrometry method, an ion electrode method, measurement of gas-phase pressure and liquid-phase temperature, and conversion from pH and alkalinity. When the amount of carbon dioxide gas varies depending on the determination method, the amount of carbon dioxide gas used is determined by measuring the gas-phase pressure and liquid-phase temperature, specifically by the method described in the Examples.

Although the embodiments are described above, it will be understood that various changes in form and details can be made without departing from the spirit and scope of the claims.

The present disclosure includes, for example, the following.

### Item 1

A method for producing a compound represented by formula (1):

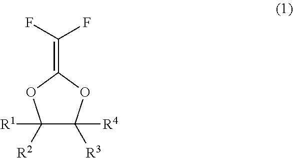

wherein R¹ to R⁴ are each independently a fluorine atom or a C1-C7 fluoroalkyl group optionally containing ethereal oxygen,
the method comprising the following steps A, B, C, and D:
step A of reacting a compound represented by formula (2):

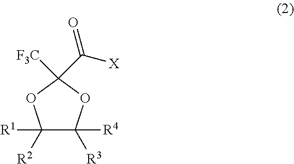

wherein X is a hydroxy group, a fluorine atom, a chlorine atom, or a C1-C3 alkoxy group in which one or more hydrogen atoms are optionally replaced by fluorine atoms, and R¹ to R⁴ are the same as above, with at least one base selected from the group consisting of hydroxides, carbonates, and alkoxides of alkali metals and alkaline earth metals to produce a compound represented by formula (3):

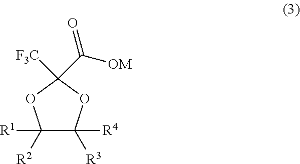

wherein M is an alkali metal atom or an alkaline earth metal atom, and R¹ to R⁴ are the same as above, thereby obtaining a reaction product having a pH range of more than 11.0;
step B of adding carbon dioxide gas to the reaction product obtained in step A, or a liquid obtained by mixing the reaction product with water, to adjust the pH to 6.0 to 11.0, thereby obtaining a pH-adjusted liquid;
step C of concentrating the pH-adjusted liquid obtained in step B to obtain a concentrate; and
step D of heating the concentrate obtained in step C to thermally decompose the compound represented by formula (3), thereby producing the compound represented by formula (1).

### Item 2

The production method according to Item 1, wherein R¹ to R⁴ are each independently a fluorine atom, a perfluoro C1-C7 alkyl group, or a perfluoro C1-C7 alkoxy group.

### Item 3

The production method according to Item 1 or 2, wherein R¹ is a trifluoromethyl group or a fluorine atom, and R² to R⁴ are all fluorine atoms.

### Item 4

The production method according to any one of Items 1 to 3, wherein X is a hydroxy group, a fluorine atom, a chlorine atom, methoxy, ethoxy, n-propoxy, i-propoxy, trifluoromethoxy, or 2,2,2-trifluoroethoxy.

### Item 5

The production method according to any one of Items 1 to 4, wherein the base is at least one compound selected from the group consisting of potassium carbonate, sodium carbonate, magnesium carbonate, sodium hydrogen carbonate, potassium hydrogen carbonate, potassium hydroxide, sodium hydroxide, magnesium hydroxide, potassium methoxide, sodium methoxide, magnesium methoxide, potassium ethoxide, sodium ethoxide, and magnesium ethoxide.

### Item 6

The production method according to any one of Items 1 to 5, wherein the pH range of 6.0 to 11.0 in step B is 6.0 to 10.0.

### Item 7

The production method according to any one of Items 1 to 6, wherein the amount of carbon dioxide gas added in step B is 100 to 10000 mol % based on the content of the compound represented by formula (3) in the reaction product obtained in step A.

### Item 8

The production method according to any one of Items 1 to 7, wherein steps C and D are performed continuously in the same reactor.

### Item 9

The production method according to any one of Items 1 to 8, wherein
R¹ is a trifluoromethyl group or a fluorine atom,
R² to R⁴ are all fluorine atoms,
X is a fluorine atom, methoxy, ethoxy, n-propoxy, or i-propoxy, the base is at least one compound selected from the group consisting of potassium carbonate, sodium carbonate, potassium hydroxide, and sodium hydroxide,
M is a potassium atom or a sodium atom,
the pH range of 6.0 to 11.0 in step B is 6.5 to 8.5, and
the amount of carbon dioxide gas added in step B is 500 to 6000 mol % based on the content of the compound represented by formula (3) in the reaction product obtained in step A.

### Item 10

The production method according to any one of Items 1 to 9, wherein
R¹ is a trifluoromethyl group or a fluorine atom,
R² to R⁴ are all fluorine atoms,
X is a fluorine atom or methoxy,
the base is at least one compound selected from the group consisting of potassium carbonate and potassium hydroxide, M is a potassium atom,
the pH range of 6.0 to 11.0 in step B is 6.9 to 7.9, and
the amount of carbon dioxide gas added in step B is 600 to 5000 mol % based on the content of the compound represented by formula (3) in the reaction product obtained in step A.

### Item 11

A composition comprising:
at least one compound selected from the group consisting of a compound represented by formula (3):

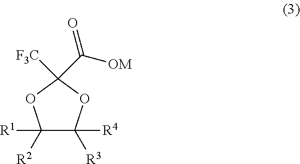

wherein R¹ to R⁴ are each independently a fluorine atom or a C1-C7 fluoroalkyl group optionally containing ethereal oxygen, and M is an alkali metal atom or an alkaline earth metal atom; and a compound represented by formula (4):

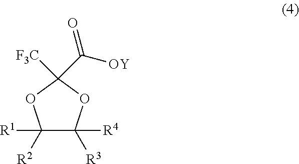

wherein Y is a hydrogen atom or a C1-C3 alkyl group in which one or more hydrogen atoms are optionally replaced by fluorine atoms, and R¹ to R⁴ are the same as above; and
carbon dioxide gas,
   wherein when the composition is an aqueous solution of at least one compound selected from the group consisting of the compounds represented by formulas (3) and (4), its pH is within the range of 6.0 to 11.0, and when the composition is a non-aqueous solution, the pH of an aqueous solution obtained by adding water to the composition is within the range of 6.0 to 11.0.

### Item 12

The composition according to Item 11, further comprising at least one base selected from the group consisting of hydroxides, carbonates, and alkoxides of alkali metals and alkaline earth metals.

### Item 13

The composition according to Item 11 or 12, wherein R¹ to R⁴ are each independently a fluorine atom, a perfluoro C1-C7 alkyl group, or a perfluoro C1-C7 alkoxy group.

### Item 14

The composition according to any one of Items 11 to 13, wherein R¹ is a trifluoromethyl group or a fluorine atom, and R² to R⁴ are all fluorine atoms.

### Item 15

The composition according to any one of Items 11 to 14, wherein Y is a hydrogen atom, methyl, ethyl, n-propyl, i-propyl, trifluoromethyl, or 2,2,2-trifluoroethyl.

### Item 16

The composition according to any one of Items 12 to 15, wherein the base is at least one compound selected from the group consisting of potassium carbonate, sodium carbonate, magnesium carbonate, sodium hydrogen carbonate, potassium hydrogen carbonate, potassium hydroxide, sodium hydroxide, magnesium hydroxide, potassium methoxide, sodium methoxide, magnesium methoxide, potassium ethoxide, sodium ethoxide, and magnesium ethoxide.

### Item 17

The composition according to any one of Items 11 to 16, wherein the pH of the aqueous solution is within the range of 6.0 to 10.0.

### Item 18

The composition according to any one of Items 11 to 16, wherein the pH of the aqueous solution is within the range of 6.5 to 10.0.

### Item 19

The composition according to any one of Items 12 to 18, wherein
R¹ is a trifluoromethyl group or a fluorine atom,
R² to R⁴ are all fluorine atoms,
the base is at least one compound selected from the group consisting of potassium carbonate, sodium carbonate, potassium hydroxide, and sodium hydroxide,
Y is methyl, ethyl, n-propyl, or i-propyl,
M is a potassium atom or a sodium atom,
the aqueous solution has a pH within the range of 6.5 to 8.5, and
the content of carbon dioxide gas is 0.2 to 30 mass % based on the mass of the composition.

### Item 20

The composition according to any one of Items 12 to 19, wherein

R¹ is a trifluoromethyl group or a fluorine atom,
R² to R⁴ are all fluorine atoms,
the base is at least one compound selected from the group consisting of potassium carbonate and potassium hydroxide, Y is methyl,
M is a potassium atom,
the aqueous solution has a pH within the range of 6.9 to 7.9, and
the content of carbon dioxide gas is 0.3 to 17 mass % based on the mass of the composition.

### Item 21

A composition comprising:
at least one compound selected from the group consisting of a compound represented by formula (3):

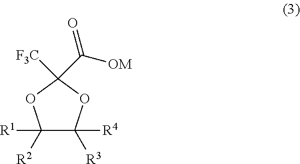

wherein R¹ to R⁴ are each independently a fluorine atom or a C1-C7 fluoroalkyl group optionally containing ethereal oxygen, and M is an alkali metal atom or an alkaline earth metal atom; and a compound represented by formula (4):

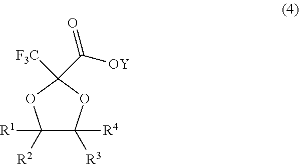

wherein Y is a hydrogen atom or a C1-C3 alkyl group in which one or more hydrogen atoms are optionally replaced by fluorine atoms, and R¹ to R⁴ are the same as above; and
carbon dioxide gas,
   wherein the content of carbon dioxide gas is 0.05 to 1000 mass % based on the mass of the composition.

### Item 22

The composition according to Item 21, wherein the content of carbon dioxide gas is 0.1 to 500 mass % based on the mass of the composition.

### Item 23

The composition according to Item 21, wherein the content of carbon dioxide gas is 0.1 to 100 mass % based on the mass of the composition.

### Item 24

The composition according to any one of Items 21 to 23, wherein R¹ to R⁴ are each independently a fluorine atom, a perfluoro C1-C7 alkyl group, or a perfluoro C1-C7 alkoxy group.

### Item 25

The composition according to any one of Items 21 to 24, wherein R¹ is a trifluoromethyl group or a fluorine atom, and R² to R⁴ are all fluorine atoms.

### Item 26

The composition according to any one of Items 21 to 25, wherein Y is a hydrogen atom, methyl, ethyl, n-propyl, i-propyl, trifluoromethyl, or 2,2,2-trifluoroethyl.

### Item 27

The composition according to any one of Items 21 to 26, further comprising a base, wherein
R¹ is a trifluoromethyl group or a fluorine atom,
R² to R⁴ are all fluorine atoms,
the base is at least one compound selected from the group consisting of potassium carbonate, sodium carbonate, potassium hydroxide, and sodium hydroxide,
Y is methyl, ethyl, n-propyl, or i-propyl,
M is a potassium atom or a sodium atom,
the aqueous solution has a pH within the range of 6.5 to 8.5, and
the content of carbon dioxide gas is 0.2 to 30 mass % based on the mass of the composition.

### Item 28

The composition according to any one of Items 21 to 27, further comprising a base, wherein
R¹ is a trifluoromethyl group or a fluorine atom,
R² to R⁴ are all fluorine atoms,
the base is at least one compound selected from the group consisting of potassium carbonate and potassium hydroxide,
Y is methyl,
M is a potassium atom,
the aqueous solution has a pH within the range of 6.9 to 7.9, and
the content of carbon dioxide gas is 0.3 to 17 mass % based on the mass of the composition.

### EXAMPLES

An embodiment of the present disclosure is described in more detail below with Examples; however, the present disclosure is not limited thereto.

In the Examples etc., the pH measurement was performed by using a Benchtop pH/Water Quality Analyzer F-74, produced by Horiba, Ltd. The amount of carbon dioxide gas was calculated from the measurement of gas-phase pressure and liquid-phase temperature and expressed as the amount (mol %) relative to the amount of compound (3). Specifically, the amount of carbon dioxide gas was determined by subtracting the amount of carbon dioxide gas when the temperature and pressure in the reactor became roughly constant after introduction, from the initial amount of carbon dioxide gas introduced into the reactor (amount of carbon dioxide gas added). The latter amount of carbon dioxide gas was calculated by measuring the temperature, pressure, and space volume inside the reaction vessel.

The symbols and abbreviations in the Examples etc. are used with the following meanings.
GC: gas chromatography
Compounds a to e: compounds represented by the following formulas:

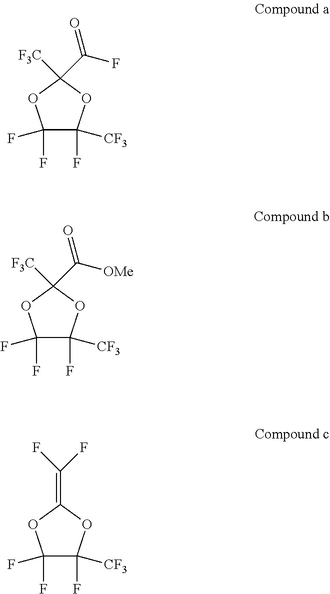 [SMILES: O=C(F)C1(C(F)(F)F)OC(F)(F)C(F)(C(F)(F)F)O1 ; COC(=O)C1(C(F)(F)F)OC(F)(F)C(F)(C(F)(F)F)O1 ; FC(F)=C1OC(F)(F)C(F)(C(F)(F)F)O1]

[symbol] Compound d Compound e

### Production Example 1: Production of Compounds a and b

A crude product (purity: 95%) containing compound a was synthesized according to the description in Reference Example 1 of PTL 3, and a crude product (purity: 98%) containing compound b was synthesized according to the description in Example 5 of PTL 1. They were used as raw materials for Comparative Examples and Examples.

### Comparative Example 1

12.1 g (purity: 95%) of the crude product containing compound a was added to a reaction vessel, and 5.3 g of potassium carbonate and 10 g of water were further added, followed by stirring at 0 to 10° C. for 3 hours to obtain a reaction liquid. The reaction liquid was filtered, concentrated, and analyzed by NMR, revealing that the concentrate contained 12.6 g of compound e (yield: 98%). When 1 g of the concentrate was dissolved in 50 g of water and the pH was measured, the pH was 12.0. The obtained concentrate was added to a reactor and heated at 200° C. for 4 hours and then at 300° C. for 1 hour. The product was collected in a −78° C. trap and analyzed by NMR and GC. As a result, a product containing compound c was obtained in a yield of 63% (molar ratio of compound c to compound d=88:12).

### Comparative Example 2

41 g (purity: 98%) of the crude product containing compound b was added to a reaction vessel, and 7.4 g of potassium hydroxide and 42 g of methanol were further added, followed by stirring at 20° C. for 1 hour to obtain a reaction liquid. The reaction liquid was filtered, concentrated, and analyzed by NMR, revealing that the concentrate contained 42.7 g of compound e (yield: 95%). When 1 g of the concentrate was dissolved in 50 g of water and the pH was measured, the pH was 13.6. The obtained concentrate was added to a reactor and heated at 200° C. for 4 hours and at 300° C. for 1 hour. The product was collected in a −78° C. trap and analyzed by NMR and GC. As a result, a product containing compound c was obtained in a yield of 72% (molar ratio of compound c to compound d=90:10).

### Reference Example 1

12.1 g (purity: 95%) of the crude product containing compound a was added to a reaction vessel, and 6.2 g of potassium carbonate and 10 g of water were further added, followed by stirring at 0 to 10° C. for 3 hours to obtain a reaction liquid. The entire reaction liquid was filtered, and the pH was confirmed to be 12.0. Hydrofluoric acid was added to the reaction liquid to prepare a pH-adjusted liquid with a pH of 7.9 (composition (A): a composition containing compound e, potassium carbonate, KF, and water). The pH-adjusted liquid was filtered and concentrated to obtain a concentrate. The obtained concentrate was analyzed by NMR and found to contain 12.6 g of compound e (yield: 98%). Upon inspection of the inside of the SUS reactor after the reaction, it was confirmed that the surface of the reactor was corroded. The obtained concentrate was added to a reactor and vacuum-dried at 140° C. for 15 hours. After it was confirmed to be highly concentrated (composition (B): a composition containing compound e, potassium carbonate, KF, and water (97 ppm)), the temperature was raised to 300° C., and heating was further performed at 300° C. for 1 hour. The product was collected in a −78° C. trap. As a result of analyzing the product by NMR and GC, the product containing compound c was obtained in a yield of 70% (molar ratio of compound c to compound d=>99:<1).

### Reference Example 2

41.0 g (purity: 98%) of the crude product containing compound b was added to a reaction vessel, and 8.4 g of potassium hydroxide and 42 g of methanol were further added, followed by stirring at 20° C. for 1 hour to obtain a reaction liquid. The entire reaction liquid was filtered, 1 g of the reaction liquid was added to 10 g of water, and the mixture was vigorously stirred and then allowed to stand. The pH of the aqueous phase was confirmed to be 12.0. Hydrofluoric acid was added to the reaction liquid to prepare a pH-adjusted liquid with a pH of 7.5 (composition (C): a composition containing compound b (2.0 GC %), compound e, potassium hydroxide, and water). The pH-adjusted liquid was filtered and concentrated to obtain a concentrate. The obtained concentrate was analyzed by NMR and found to contain 42.8 g of compound e (yield: 95%). Upon inspection of the inside of the SUS reactor after the reaction, it was confirmed that the surface of the reactor was corroded. The obtained product was added to a reactor and vacuum-dried at 140° C. for 15 hours. After it was confirmed to be highly concentrated (composition (D): a composition containing compound e, potassium hydroxide, and water (54 ppm)), the temperature was raised to 300° C., and heating was further performed at 300° C. for 1 hour. The product was collected in a −78° C. trap. As a result of analyzing the product by NMR and GC, the product containing compound c was obtained in a yield of 80% (molar ratio of compound c to compound d=>99:<1).

### Example 1

11.5 g (purity: 95%) of the crude product containing compound a was added to a reaction vessel, and 5.5 g of potassium carbonate and 10 g of water were further added, followed by stirring at 0 to 10° C. for 3 hours to obtain a reaction liquid. The entire reaction liquid was filtered, and the pH was confirmed to be 12.0. The filtrate was transferred to a 2.0 L SUS pressure vessel, after which 8.9 g (600 mol %) of carbon dioxide gas was added, followed by stirring for 1 hour to prepare a pH-adjusted liquid with a pH of 7.9. At this time, the temperature inside the reaction vessel was 25° C., and the pressure was 0.25 MPaG. The obtained pH-adjusted liquid was concentrated, and the concentrate was analyzed by NMR and found to contain 11.6 g of compound e (yield: 95%). After the reaction, the inside of the SUS reactor was inspected, but no corrosion was found on the surface of the reactor. The obtained concentrate was added to a reactor and vacuum-dried at 140° C. for 15 hours (step C). After it was confirmed to be highly concentrated (water content: 100 ppm or less), the temperature was raised to 300° C., and heating was further performed at 300° C. for 1 hour (step (D)). The product was collected in a-78° C. trap. As a result of analyzing the product by NMR and GC, the product containing compound c was obtained in a yield of 78% (molar ratio of compound c to compound d=>99:<1).

The mass of the pH-adjusted liquid (compositions (1) and (2)) was 27 g, and its formulation was as follows: compound e: 11.6 g, water: 10 g, carbon dioxide gas: 0.09 g (molar ratio relative to compound e: 6 mol %, 0.3 mass % relative to the mass of the composition), potassium fluoride: 1.9 g, and a small amount of potassium carbonate. The amount of carbon dioxide gas was determined as follows using the reaction vessel volume (2 L), the amount of carbon dioxide gas added (8.9 g; 600 mol %), the temperature inside the reaction vessel (25° C.), and the pressure inside the reaction vessel (0.25 MPaG). This determination method is the same in other examples.

In gas equation of state: PV=nRT,
P: 0.25 [MPaG]
V: 1.985 [L] (calculated by subtracting the content volume from the reaction vessel capacity)
Content volume: volume of solvent (weight used/specific gravity)+volume of compound e (weight produced/specific gravity: 2.5 g/mL)+volume of compound b (weight produced/specific gravity: 1.7 g/mL)

The specific gravity of the solvent is 1.0 g/mL for water and 0.78 g/mL for methanol.
n: Amount of carbon dioxide gas in the gas phase to be determined [mol]
R: 8.31×10^{3 [}Pa·L/(mol·K)]
T: 298 [K] (25 [° C.])

The gas equation of state is rearranged to n=PV/RT. n=(0.25×10⁶×(2.0−(10/1+11.6/2.5)/1000))/(8.31×10³×298)=0.199 The amount of carbon dioxide gas in the gas phase (0.199 mol) was subtracted from the amount of carbon dioxide gas added (8.9 g (0.201 mol)) to determine the amount of carbon dioxide gas in the composition (0.201−0.199=0.002 mol (0.09 g)).

### Example 2

11.0 g (purity: 95%) of the crude product containing compound a was added to a reaction vessel, and 5.5 g of potassium carbonate and 10 g of water were further added, followed by stirring at 0 to 10° C. for 3 hours to obtain a reaction liquid. The entire reaction liquid was filtered, and the pH was confirmed to be 12.0. The filtrate was transferred to a 2.0 L SUS pressure vessel, after which 21.4 g (1500 mol %) of carbon dioxide gas was added, followed by stirring for 1 hour to prepare a pH-adjusted liquid with a pH of 7.4. At this time, the temperature inside the reaction vessel was 25° C., and the pressure was 0.60 MPaG. The obtained pH-adjusted liquid was concentrated, and the concentrate was analyzed by NMR and found to contain 11.2 g of compound e (yield: 96%). After the reaction, the inside of the SUS reactor was inspected, but no corrosion was found on the surface of the reactor. The obtained concentrate was added to a reactor and vacuum-dried at 140° C. for 15 hours (step C). After it was confirmed to be highly concentrated (water content: 100 ppm or less), the temperature was raised to 300° C., and heating was further performed at 300° C. for 1 hour (step (D)). The product was collected in a −78° C. trap. As a result of analyzing the product by NMR and GC, the product containing compound c was obtained in a yield of 78% (molar ratio of compound c to compound d=>99:<1).

The mass of the pH-adjusted liquid (compositions (1) and (2)) was 27 g, and its formulation was as follows: compound e: 11.2 g, water: 10 g, carbon dioxide gas: 0.24 g (molar ratio relative to compound e: 17 mol %, 0.9 mass % relative to the mass of the composition), potassium fluoride: 1.8 g, and a small amount of potassium carbonate.

### Example 3

11.2 g (purity: 95%) of the crude product containing compound a was added to a reaction vessel, and 5.5 g of potassium carbonate and 10 g of water were further added, followed by stirring at 0 to 10° C. for 3 hours to obtain a reaction liquid. The entire reaction liquid was filtered, and the pH was confirmed to be 12.0. The filtrate was transferred to a 2.0 L SUS pressure vessel, after which 74.2 g (5000 mol %) of carbon dioxide gas was added, followed by stirring for 1 hour to prepare a pH-adjusted liquid with a pH of 6.9. At this time, the temperature inside the reaction vessel was 25° C., and the pressure was 2.08 MPaG. The obtained pH-adjusted liquid was concentrated, and the concentrate was analyzed by NMR and found to contain 11.7 g of compound e (yield: 98%). After the reaction, the inside of the SUS reactor was inspected, but no corrosion was found on the surface of the reactor. The obtained concentrate was added to a reactor and vacuum-dried at 140° C. for 15 hours (step C). After it was confirmed to be highly concentrated (water content: 100 ppm or less), the temperature was raised to 300° C., and heating was further performed at 300° C. for 1 hour (step (D)). The product was collected in a −78° C. trap. As a result of analyzing the product by NMR and GC, the product containing compound c was obtained in a yield of 82% (molar ratio of compound c to compound d=>99:<1).

The mass of the pH-adjusted liquid (compositions (1) and (2)) was 28 g, and its formulation was as follows: compound e: 11.7 g, water: 10 g, carbon dioxide gas: 0.96 g (molar ratio relative to compound e: 65 mol %, 3 mass % relative to the mass of the composition), potassium fluoride: 1.9 g, and a small amount of potassium carbonate.

### Example 4

43.0 g (purity: 98%) of the crude product containing compound b was added to a reaction vessel, and 8.1 g of potassium hydroxide and 42 g of methanol were further added, followed by stirring at 20° C. for 1 hour to obtain a reaction liquid. The entire reaction liquid was filtered, 1 g of the filtrate was added to 10 g of water, and the mixture was vigorously stirred and then allowed to stand. The pH of the aqueous phase was confirmed to be 13.6. The filtrate was transferred to a 10 L SUS pressure vessel, after which 32.3 g (600 mol %) of carbon dioxide gas was added, followed by stirring for 1 hour to prepare a pH-adjusted liquid with a pH of 7.9. At this time, the temperature inside the reaction vessel was 25° C., and the pressure was 0.17 MPaG. The obtained pH-adjusted liquid was concentrated, and the concentrate was analyzed by NMR and found to contain 42.3 g of compound e (yield: 93%). After the reaction, the inside of the SUS reactor was inspected, but no corrosion was found on the surface of the reactor. The obtained concentrate was added to a reactor and vacuum-dried at 140° C. for 15 hours (step C). After it was confirmed to be highly concentrated (water content: 100 ppm or less), the temperature was raised to 300° C., and heating was further performed at 300° C. for 1 hour (step (D)). The product was collected in a −78° C. trap. As a result of analyzing the product by NMR and GC, the product containing compound c was obtained in a yield of 79% (molar ratio of compound c to compound d=>99:<1).

The mass of the pH-adjusted liquid (compositions (1) and (2)) was 95 g, and its formulation was as follows: compound e: 42.3 g, compound b: 0.76 g, methanol: 45.8 g, carbon dioxide gas: 2.3 g (molar ratio relative to compound e: 42 mol %, 2 mass % relative to the mass of the composition), potassium fluoride: 0.7 g, a small amount of potassium carbonate, and a small amount of water.

### Example 5

44.5 g (purity: 98%) of the crude product containing compound b was added to a reaction vessel, and 8.3 g of potassium hydroxide and 42 g of methanol were further added, followed by stirring at 20° C. for 1 hour to obtain a reaction liquid. The entire reaction liquid was filtered, 1 g of the filtrate was added to 10 g of water, and the mixture was vigorously stirred and then allowed to stand. The pH of the aqueous phase was confirmed to be 13.8. The filtrate was transferred to a 10 L SUS pressure vessel, after which 81.5 g (1500 mol %) of carbon dioxide gas was added, followed by stirring for 1 hour to prepare a pH-adjusted liquid with a pH of 7.4. At this time, the temperature inside the reaction vessel was 25° C., and the pressure was 0.43 MPaG. The obtained pH-adjusted liquid was concentrated, and the concentrate was analyzed by NMR and found to contain 42.7 g of compound e (yield: 91%). After the reaction, the inside of the SUS reactor was inspected, but no corrosion was found on the surface of the reactor. The obtained concentrate was added to a reactor and vacuum-dried at 140° C. for 15 hours (step C). After it was confirmed to be highly concentrated (water content: 100 ppm or less), the temperature was raised to 300° C., and heating was further performed at 300° C. for 1 hour (step (D)). The product was collected in a −78° C. trap. As a result of analyzing the product by NMR and GC, the product containing compound c was obtained in a yield of 80% (molar ratio of compound c to compound d=>99:<1).

The mass of the pH-adjusted liquid (compositions (1) and (2)) was 100 g, and its formulation was as follows: compound e: 42.7 g, compound b: 0.65 g, methanol: 45.9 g, carbon dioxide gas: 5.7 g (molar ratio relative to compound e: 105 mol %, 6 mass % relative to the mass of the composition), potassium fluoride: 0.7 g, a small amount of potassium carbonate, and a small amount of water.

### Example 6

46.0 g (purity: 98%) of the crude product containing compound b was added to a reaction vessel, and 8.0 g of potassium hydroxide and 42 g of methanol were further added, followed by stirring at 20° C. for 1 hour to obtain a reaction liquid. The reaction liquid was filtered, 1 g of the filtrate was added to 10 g of water, and the mixture was vigorously stirred and then allowed to stand. The pH of the aqueous phase was confirmed to be 13.6. The filtrate was transferred to a 10 L SUS pressure vessel, after which 277.4 g (5000 mol %) of carbon dioxide gas was added, followed by stirring for 1 hour to prepare a pH-adjusted liquid with a pH of 6.9. At this time, the temperature inside the reaction vessel was 25° C., and the pressure was 1.46 MPaG. The obtained pH-adjusted liquid was concentrated, and the concentrate was analyzed by NMR and found to contain 43.6 g of compound e (yield: 90%). After the reaction, the inside of the SUS reactor was inspected, but no corrosion was found on the surface of the reactor. The obtained concentrate was added to a reactor and vacuum-dried at 140° C. for 15 hours (step C). After it was confirmed to be highly concentrated (water content: 100 ppm or less), the temperature was raised to 300° C., and heating was further performed at 300° C. for 1 hour (step (D)). The product was collected in a −78° C. trap. As a result of analyzing the product by NMR and GC, the product containing compound c was obtained in a yield of 84% (molar ratio of compound c to compound d=>99:<1).

The mass of the pH-adjusted liquid (compositions (1) and (2)) was 115 g, and its formulation was as follows: compound e: 43.6 g, compound b: 0.41 g, methanol: 46.0 g, carbon dioxide gas: 19.4 g (molar ratio relative to compound e: 350 mol %, 17 mass % relative to the mass of the composition), potassium fluoride: 0.72 g, a small amount of potassium carbonate, and a small amount of water.

1. A method for producing a compound represented by formula (1): 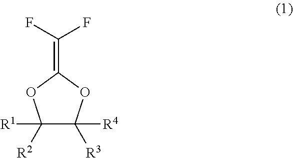
wherein R¹ to R⁴ are each independently a fluorine atom or a C1-C7 fluoroalkyl group optionally containing ethereal oxygen,
the method comprising the following steps A, B, C, and D:
step A of reacting a compound represented by formula (2): 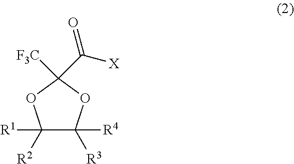
wherein X is a hydroxy group, a fluorine atom, a chlorine atom, or a C1-C3 alkoxy group in which one or more hydrogen atoms are optionally replaced by fluorine atoms, and R¹ to R⁴ are the same as above, with at least one base selected from the group consisting of hydroxides, carbonates, and alkoxides of alkali metals and alkaline earth metals to produce a compound represented by formula (3): 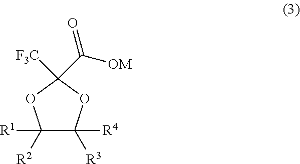
wherein M is an alkali metal atom or an alkaline earth metal atom, and R¹ to R⁴ are the same as above, thereby obtaining a reaction product having a pH range of more than 11.0;
step B of adding carbon dioxide gas to the reaction product obtained in step A, or a liquid obtained by mixing the reaction product with water, to adjust the pH to 6.0 to 11.0, thereby obtaining a pH-adjusted liquid;
step C of concentrating the pH-adjusted liquid obtained in step B to obtain a concentrate; and
step D of heating the concentrate obtained in step C to thermally decompose the compound represented by formula (3), thereby producing the compound represented by formula (1).

2. The production method according to claim 1, wherein R¹ to R⁴ are each independently a fluorine atom, a perfluoro C1-C7 alkyl group, or a perfluoro C1-C7 alkoxy group.

3. The production method according to claim 1, wherein R¹ is a trifluoromethyl group or a fluorine atom, and R² to R⁴ are all fluorine atoms.

4. The production method according to claim 1, wherein X is a hydroxy group, a fluorine atom, a chlorine atom, methoxy, ethoxy, n-propoxy, i-propoxy, trifluoromethoxy, or 2,2,2-trifluoroethoxy.

5. The production method according to claim 1, wherein the base is at least one compound selected from the group consisting of potassium carbonate, sodium carbonate, magnesium carbonate, sodium hydrogen carbonate, potassium hydrogen carbonate, potassium hydroxide, sodium hydroxide, magnesium hydroxide, potassium methoxide, sodium methoxide, magnesium methoxide, potassium ethoxide, sodium ethoxide, and magnesium ethoxide.

6. The production method according to claim 1, wherein the pH range of 6.0 to 11.0 in step B is 6.0 to 10.0.

7. The production method according to claim 1, wherein the amount of carbon dioxide gas added in step B is 100 to 10000 mol % based on the content of the compound represented by formula (3) in the reaction product obtained in step A.

8. The production method according to claim 1, wherein steps C and D are performed continuously in the same reactor.

9. The production method according to claim 1, wherein
R¹ is a trifluoromethyl group or a fluorine atom,
R² to R⁴ are all fluorine atoms,
X is a fluorine atom, methoxy, ethoxy, n-propoxy, or i-propoxy,
the base is at least one compound selected from the group consisting of potassium carbonate, sodium carbonate, potassium hydroxide, and sodium hydroxide,
M is a potassium atom or a sodium atom,
the pH range of 6.0 to 11.0 in step B is 6.5 to 8.5, and
the amount of carbon dioxide gas added in step B is 500 to 6000 mol % based on the content of the compound represented by formula (3) in the reaction product obtained in step A.

10. The production method according to claim 1, wherein
R¹ is a trifluoromethyl group or a fluorine atom,
R² to R⁴ are all fluorine atoms,
X is a fluorine atom or methoxy,
the base is at least one compound selected from the group consisting of potassium carbonate and potassium hydroxide,
M is a potassium atom,
the pH range of 6.0 to 11.0 in step B is 6.9 to 7.9, and
the amount of carbon dioxide gas added in step B is 600 to 5000 mol % based on the content of the compound represented by formula (3) in the reaction product obtained in step A.

11. A composition comprising:
at least one compound selected from the group consisting of a compound represented by formula (3): 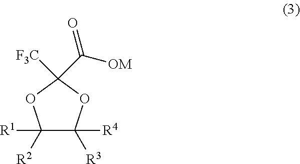
wherein R¹ to R⁴ are each independently a fluorine atom or a C1-C7 fluoroalkyl group optionally containing ethereal oxygen, and M is an alkali metal atom or an alkaline earth metal atom; and a compound represented by formula (4): 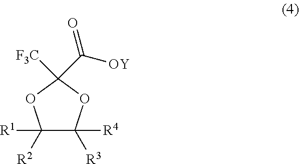
wherein Y is a hydrogen atom or a C1-C3 alkyl group in which one or more hydrogen atoms are optionally replaced by fluorine atoms, and R¹ to R⁴ are the same as above; and
carbon dioxide gas,
wherein when the composition is an aqueous solution of at least one compound selected from the group consisting of the compounds represented by formulas (3) and (4), its pH is within the range of 6.0 to 11.0, and when the composition is a non-aqueous solution, the pH of an aqueous solution obtained by adding water to the composition is within the range of 6.0 to 11.0.

12. The composition according to claim 11, further comprising at least one base selected from the group consisting of hydroxides, carbonates, and alkoxides of alkali metals and alkaline earth metals.

13. The composition according to claim 11, wherein R¹ to R⁴ are each independently a fluorine atom, a perfluoro C1-C7 alkyl group, or a perfluoro C1-C7 alkoxy group.

14. The composition according to claim 11, wherein R¹ is a trifluoromethyl group or a fluorine atom, and R² to R⁴ are all fluorine atoms.

15. The composition according to claim 11, wherein Y is a hydrogen atom, methyl, ethyl, n-propyl, i-propyl, trifluoromethyl, or 2,2,2-trifluoroethyl.

16. The composition according to claim 12, wherein the base is at least one compound selected from the group consisting of potassium carbonate, sodium carbonate, magnesium carbonate, sodium hydrogen carbonate, potassium hydrogen carbonate, potassium hydroxide, sodium hydroxide, magnesium hydroxide, potassium methoxide, sodium methoxide, magnesium methoxide, potassium ethoxide, sodium ethoxide, and magnesium ethoxide.

17. The composition according to claim 11, wherein the pH of the aqueous solution is within the range of 6.0 to 10.0.

18. The composition according to claim 11, wherein the pH of the aqueous solution is within the range of 6.5 to 10.0.

19. The composition according to claim 12, wherein
R¹ is a trifluoromethyl group or a fluorine atom,
R² to R⁴ are all fluorine atoms,
the base is at least one compound selected from the group consisting of potassium carbonate, sodium carbonate, potassium hydroxide, and sodium hydroxide,
Y is methyl, ethyl, n-propyl, or i-propyl,
M is a potassium atom or a sodium atom,
the aqueous solution has a pH within the range of 6.5 to 8.5, and
the content of carbon dioxide gas is 0.2 to 30 mass % based on the mass of the composition.

20. The composition according to claim 12, wherein
R¹ is a trifluoromethyl group or a fluorine atom,
R² to R⁴ are all fluorine atoms,
the base is at least one compound selected from the group consisting of potassium carbonate and potassium hydroxide,
Y is methyl,
M is a potassium atom,
the aqueous solution has a pH within the range of 6.9 to 7.9, and
the content of carbon dioxide gas is 0.3 to 17 mass % based on the mass of the composition.

21. A composition comprising:
at least one compound selected from the group consisting of a compound represented by formula (3): 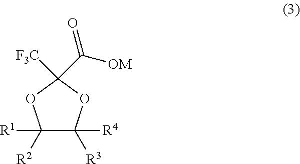
wherein R¹ to R⁴ are each independently a fluorine atom or a C1-C7 fluoroalkyl group optionally containing ethereal oxygen, and M is an alkali metal atom or an alkaline earth metal atom; and a compound represented by formula (4): 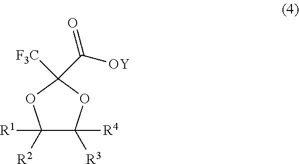
wherein Y is a hydrogen atom or a C1-C3 alkyl group in which one or more hydrogen atoms are optionally replaced by fluorine atoms, and R¹ to R⁴ are the same as above; and
carbon dioxide gas,
wherein the content of carbon dioxide gas is 0.05 to 1000 mass % based on the mass of the composition.

22. The composition according to claim 21, wherein the content of carbon dioxide gas is 0.1 to 500 mass % based on the mass of the composition.

23. The composition according to claim 21, wherein the content of carbon dioxide gas is 0.1 to 100 mass % based on the mass of the composition.

24. The composition according to claim 21, wherein R¹ to R⁴ are each independently a fluorine atom, a perfluoro C1-C7 alkyl group, or a perfluoro C1-C7 alkoxy group.

25. The composition according to claim 21, wherein R¹ is a trifluoromethyl group or a fluorine atom, and R² to R⁴ are all fluorine atoms.

26. The composition according to claim 21, wherein Y is a hydrogen atom, methyl, ethyl, n-propyl, i-propyl, trifluoromethyl, or 2,2,2-trifluoroethyl.

27. The composition according to claim 21, further comprising a base, wherein
R¹ is a trifluoromethyl group or a fluorine atom,
R² to R⁴ are all fluorine atoms,
the base is at least one compound selected from the group consisting of potassium carbonate, sodium carbonate, potassium hydroxide, and sodium hydroxide,
Y is methyl, ethyl, n-propyl, or i-propyl,
M is a potassium atom or a sodium atom,
the aqueous solution has a pH within the range of 6.5 to 8.5, and
the content of carbon dioxide gas is 0.2 to 30 mass % based on the mass of the composition.

28. The composition according to claim 21, further comprising a base, wherein
R¹ is a trifluoromethyl group or a fluorine atom,
R² to R⁴ are all fluorine atoms,
the base is at least one compound selected from the group consisting of potassium carbonate and potassium hydroxide,
Y is methyl,
M is a potassium atom,
the aqueous solution has a pH within the range of 6.9 to 7.9, and
the content of carbon dioxide gas is 0.3 to 17 mass % based on the mass of the composition.
